(19) United States
(12) Patent Application Publication
(10) Pub. No.: US 2016/0015770 A1
(43) Pub. Date: Jan. 21, 2016

(54) COMPOSITIONS FOR TREATMENT OF RETINAL DETACHMENT

(71) Applicant: THE REGENTS OF THE UNIVERSITY OF MICHIGAN, Ann Arbor, MI (US)

(72) Inventors: David N. Zacks, Ann Arbor, MI (US); Nicholas Chinskey, Ann Arbor, MI (US)

(21) Appl. No.: 14/775,439

(22) PCT Filed: Mar. 12, 2014
(86) PCT No.: PCT/US14/24673

§ 371 Date: Sep. 11, 2015

Related U.S. Application Data
(60) Provisional application No. 61/780,009, filed on Mar. 13, 2013.

(51) Int. Cl. A61K 38/05; A61K 39/395

(52) U.S. Cl. CPC A61K 38/05; A61K 39/3955

(57) ABSTRACT

Provided herein are compositions and methods for the treatment and/or prevention of retinal detachment. In particular, calpain inhibition is used to prolong autophagy and delay apoptosis associated with retinal detachment.

### CROSS-REFERENCE TO RELATED APPLICATION

The present application claims priority to U.S. Provisional Patent Application Ser. No. 61/780,009 filed Mar. 15, 2013, which is hereby incorporated by reference in its entirety.

### STATEMENT REGARDING GOVERNMENT FUNDING

This invention was made with government support under grant number R01EY020823-01 awarded by the National Institutes of Health. The government has certain rights in the invention.

### FIELD

Provided herein are compositions and methods for the treatment and/or prevention of retinal detachment. In particular, calpain inhibition is used to prolong autophagy and delay apoptosis associated with retinal detachment.

### BACKGROUND

Separation of photoreceptors from the underlying retinal pigment epithelium (RPE) is a common pathologic feature seen in many retinal diseases. In cases of rhegmatogenous or tractional retinal detachment, there are reliable ways of achieving anatomic approximation of the retina and RPE. In other disease processes, such as age-related macular degeneration (AMD), achieving the anatomic connection is more difficult and the separation is often chronic. Despite significant advances in the surgical and medical management of retina-RPE separation, patients often still have significant visual loss, largely through death of photoreceptors (1, 2). Thus, ways of increasing photoreceptor survival during periods of retina-RPE separation has the potential to be extremely beneficial to visual outcomes.

Using an experimental model of retinal detachment, it has been shown that retina-RPE separation results in the activation of the Fas-receptor and downstream caspase cascade, leading to apoptotic death of the photoreceptors (3-5). Fas mediated apoptosis also appears to play a role in photoreceptor cell death in more complex diseases, such as AMD (6). In the experimental system tested, inhibition of Fas-receptor activation results in a significant increase in the number of photoreceptors that survive prolonged periods of retina-RPE separation (7, 8).

An interesting paradox exists, however, with regards to the rate of photoreceptor cell death in cases of retina-RPE separation. Even though the RPE is the sole source for photoreceptor nutritional support, and apoptosis is activated within hours after separation, many of the photoreceptors can survive for an extended period of time. This is clinically apparent as eyes with retinal detachments affecting the central macula can be repaired up to one week following separation with recovery of near-normal vision (9, 10). This suggests that anti-apoptotic signals are also activated shortly after detachment in order to prolong photoreceptor survival. Several of these mechanisms have been described including IL-6 activation and autophagy (11, 12).

Autophagy, a form of cellular recycling up-regulated during times of metabolic stress, also undergoes up-regulation following retinal detachment (12). Its activation is, at least in part, Fas dependent, just like apoptosis Inhibition of autophagy, either pharmacologically or via knockout of the autophagy-related gene 5 (Atg5) gene, leads to elevated and more rapid apoptosis. Therefore, the same signal, Fas, activates both apoptosis and autophagy at the time of photoreceptor-RPE separation. Autophagy inhibits apoptosis, at least temporarily, until the cells eventually die (12). The cellular signals that transition the photoreceptors from autophagy to apoptosis, especially those activate at 7 days at the close of the therapeutic window, are currently unknown.

### SUMMARY

Provided herein are compositions and methods for the treatment and/or prevention of retinal detachment. In particular, calpain inhibition is used to prolong autophagy and delay apoptosis associated with retinal detachment.

In some embodiments, the present invention provides methods for preventing photoreceptor loss in response to retinal detachment comprising, administering a calpain inhibitor to a subject with retinal detachment or suspected of having retinal detachment. In certain embodiments, the present invention provides for the use of a calpain inhibitor for the prevention of photoreceptor apoptosis due to retinal detachment.

In particular embodiments, the subject has age-related macular degeneration (AMD). In some embodiments, the subject has a disease in which the detachment of the retina is chronic (e.g., AMD). In further embodiments, the subject is further administered a VEGF inhibitor. In additional embodiments, the VEGF inhibitor comprises an anti-VEGF antibody. In additional embodiments, the calpain inhibitor comprises calpeptin or derivative thereof. In other embodiments, the calpain inhibitor comprises MDG-28170. In additional embodiments, the calpain inhibitor comprises Calpain Inhibitor I (MG-101). In some embodiments, the calpain inhibitor is administered during or immediately after eye surgery.

### DESCRIPTION OF THE FIGURES

FIG. 1: Atg5 activity following retinal detachment and Fas activation of 661W cells. (A) Protein from attached retinas and those detached for 1, 3 and 7 days were analyzed by western blot. Actin was used as a protein loading control. There was an increase in LC3-II, Atg5 and Atg5-12, peaking at the 1 and 3 day time points. (B) The attached retina and 3 and 7 day detached samples were again compared. Baf-a or equal volume of DMSO was injected into the subretinal space at the time of detachment. There was no significant difference in Atg5-12 at 7 days with Baf-a added. (C) 661W cells were treated with Fas-activating antibody. Whole cell lysates were collected at various time points, listed by number of hours following Fas activation. Both LC3-II and Atg5-12 peaked at 12 hours. Actin served as a loading control.

FIG. 2: Calpain 1 activity following retinal detachment and Fas activation. (A) Whole retinas were harvested 3, 7 and 14 days after detachments were created. Attached retina served as controls. Protein was assayed by western blot to detect full length calpain 1 and its autolysis activated subunits. Both the 80 kd and 78 kd proteins appeared to peak about 7 days. GAPDH was used a loading control for certain experiments. (B) 661W cells were treated for 6, 12, 24 and 48 hours in Fas-activating antibody. Cells not treated with Fas served as controls. They were assayed for calpain 1 and its autolysis activated subunits, which peaked at 24 hours. Actin was used as a loading control. (C) Whole retinas samples were collected at 7 days following detachment and assayed for calpain 1 specific alpha-spectrin cleavage products. Levels were up at 7 days compared to attached retinas. (D) 661W cells treated with Fas activating antibody for 24 hours. Western blot showed an increase in total calpain 1 specific alpha-spectrin cleavage products compared to untreated cells.

FIG. 3: Calpain inhibition in 661W cells with Fas activation. (A) Cells were treated for 24 hours with Fas activating antibody and various calpain inhibitors at two concentrations. Calpain inhibitors were added 1 hour prior to Fas activating antibody. All caused a drop in cleaved alpha-spectrin breakdown product, with calpeptin causing the largest effect. Actin was used as a loading control for all experiments. (B) Similar samples were assayed for LC3-II, the most reliable indicator of autophagy. LC3-II was up at both concentrations at 24 hours after Fas activation. (C) Cells were treated at various concentrations of calpeptin for 24 hours. Both LC3-II and Atg5-12 complex peaked at the 25 uM levels. (D) 661W cells were treated with 25 mM of calpeptin for various lengths of time (in hours). There was an increase in both Atg5-12 and LC3-II following Fas-activation. This signal lasted for an extended period of time with calpeptin when compared to vehicle (DMSO) alone.

FIG. 4: Calpain inhibition following retinal detachment. Retinas were harvested 7 days following detachment. Attached retinas served as controls. Either 0.2 μg of calpeptin or vehicle (DMSO) was injected into the subretinal space at the time of detachment. Protein was assayed by western blot. GAPDH severed as a loading control. (A) There was significantly less calpain 1 specific alpha-spectrin at 7 days with calpeptin. (B) At 7 days, there was an increase in Atg5 with calpain inhibition when compared to detached retinas alone.

FIG. 5: Caspase 8 activity following Fas activation. 661W cells were treated with Fas activating antibody for 48 hours. Calpeptin was added 1 hour prior to treatment. Cells were then assayed for caspase 8 activity. Fas activation caused a significant elevation in caspase 8 activity. While 1 μM of calpeptin had little effect, both the 10 and 25 μM concentrations returned caspase 8 activity to baseline levels. Calpeptin alone had no effect on cells that were not treated with Fas activating antibody. (p<0.5; #).

### DETAILED DESCRIPTION

Provided herein are compositions and methods for the treatment and/or prevention of retinal detachment. In particular, calpain inhibition is used to prolong autophagy and delay apoptosis associated with retinal detachment.

It was discovered, during the development of embodiments of the technology describe herein, that a regulator of the shift from autophagy to apoptosis is calpain activation. Thus, calpain inhibitors find use in the treatment and prevention of cellular damage associated with retinal detachment.

Calpain inhibitors have been extensively studied and have shown to be neuro-protective in a number of models including retinal hypoxia (15), photoreceptor degeneration (16), optic neuritis and other optic nerve injuries (17-19), cataracts (20), traumatic brain injury (21), and glutamate excitotoxicity (22).

Experiments describe in the Example section below show that calpain activation after retina-RPE separation results in decreased autophagy and increased photoreceptor apoptosis. This was demonstrated in a well-established in vivo model of retinal detachment and in an in vitro cell culture model. The findings show that Atg5 undergoes up-regulation within 1 day after detachment, similar to other autophagy markers. Calpain 1 reaches peak protein and activity levels at 7 days post-detachment, where autophagy decreases. Calpain inhibitors prolong the period of autophagy, which leads to decreased apoptosis. This 7 day time period of decreased Atg5 correlates well with the clinical knowledge that retinal detachments affecting the central macula need to be fixed by 7 days to preserve quality central vision. Prior to the discoveries described herein, there has been no explanation as to why vision starts decreasing at this point. Thus, provided herein are new approaches for prolonging autophagy and decreasing apoptosis following retina-RPE separation.

Photoreceptor-RPE separation is a common feature of many retinal diseases, and the resultant photoreceptor cell death contributes greatly to the visual morbidity in these patients. Previously, it has been shown that both cell survival and cell destructive pathways are activated soon after photoreceptor-RPE separation, leading to a molecular “tug-of-war” pulling the cell towards survival or death, respectively (11-12). In other words, the photoreceptors are triggered to undergo apoptosis as their supply of nutrition is taken away. However, the cells evolved ways of stopping that terminal program for at least a short period of time, in order to recover if the stress is only temporary. It is shown herein that inhibition of calpain activation can shift the molecular cascades towards the pro-survival autophagy pathways, with fewer photoreceptors entering the apoptotic cascade.

Strategies that provide prolong survival pathways have enormous clinical implications. It is known that after 7 days of detachment, photoreceptors die and visual acuity starts to decrease (9-10). When patients present early with simple retinal detachments, they can be fixed surgically with good visual outcomes. However, in diseases such as AMD, where the detachments are chronic and approximation is not easily achieved, there needs to be ways of prolonging the survival of the remaining photoreceptors if there is any hope of improving visual outcomes.

It is shown herein that the inhibition of the calpain family, including calpain 1, prolongs autophagy. Both in the in vitro and in vivo system extended presence of LC3-II and Atg5 with calpain inhibitors was shown. The in vitro system showed that this extension of autophagy correlated with reduction of apoptosis, rescuing the cells from death.

In some embodiments, calpain inhibitors are used as an adjunct to retinal detachment surgery or anti-VEGF therapy for AMD, for the purpose of increasing final visual outcomes in these debilitating diseases.

The use of any of a number of calpain inhibitors is contemplated for use in the methods herein. Such inhibitors include, but are not limited to, acetyl-calpastatin, MDL 28170, MG 101, aclacinomycin A (aclarubicin), penicillide, ALLM, ALLN, calpain inhibitors I, III, IV, V, VI, X, XI, and XII, ALLN peptide, calpain Inhibitor XII, calpeptin, calpastatin peptide, EST, E-64-c, E-64-c, leupeptin hemisulfate, E-64, MDL-28170, MG-132 [Z-Leu-Leu-Leu-CHO], PD 145305, PD 150606, PD 151746, PGP-4008, gamma-secretase Inhibitor II, Z-L-Abu-CONH-ethyl, and Z-L-Abu-CONH(CH2)3-morpholine (available from Santa Cruz Biotechnology, Inc., TOCRIS Bioscience, and EMD Millipore).

The compounds may be administered in the form of a composition which is formulated with a pharmaceutically acceptable carrier and optional excipients, adjuvants, etc. in accordance with good pharmaceutical practice. The composition may be in the form of a solid, semi-solid or liquid dosage form: such as powder, solution, elixir, syrup, suspension, cream, drops, paste and spray. As those skilled in the art would recognize, depending on the chosen route of administration (e.g. eye drops, injection, etc.), the composition form is determined. In general, it is preferred to use a unit dosage form of the inventive inhibitor in order to achieve an easy and accurate administration of the active pharmaceutical compound. In general, the therapeutically effective pharmaceutical compound is present in such a dosage form at a concentration level ranging from about 0.5% to about 99% by weight of the total composition: i.e., in an amount sufficient to provide the desired unit dose. In some embodiments, the pharmaceutical composition may be administered in single or multiple doses. The particular route of administration and the dosage regimen will be determined by one of skill in keeping with the condition of the individual to be treated and said individual's response to the treatment. In some embodiments, a composition in a unit dosage form for administration to a subject, comprises a pharmaceutical compound and one or more nontoxic pharmaceutically acceptable carriers, adjuvants or vehicles. The amount of the active ingredient that may be combined with such materials to produce a single dosage form will vary depending upon various factors, as indicated above. A variety of materials can be used as carriers, adjuvants and vehicles in the composition of the invention, as available in the pharmaceutical art. Injectable preparations, such as oleaginous solutions, suspensions or emulsions, may be formulated as known in the art, using suitable dispersing or wetting agents and suspending agents, as needed.

In some embodiments, provided herein are compositions, kits, systems, and/or methods to prevent, inhibit, block, and/or reduce photoreceptor cell death (e.g., in a human subject in need thereof). In some embodiments, provided herein are methods and compositions that prevent apoptosis of photoreceptors. In some embodiments, photoreceptor death and/or apoptosis is caused by retinal detachment, age-related macular degeneration, trauma, cancer, tumor, inflammation, uveitis, diabetes, hereditary retinal degeneration, and/or a disease affecting photoreceptor cells. In some embodiments, photoreceptor death and/or apoptosis is caused by retinal detachment. In some embodiments, retinal detachment is caused by one or more underlying diseases, disorders, or conditions (e.g., age-related macular degeneration, trauma, cancer, tumor, inflammation, uveitis, diabetes, hereditary retinal degeneration, etc.). In some embodiments, the present invention finds utility in enhancing photoreceptor viability and/or inhibiting photoreceptor death in a variety of conditions and/or diseases including, but not limited to macular degeneration (e.g. dry, wet, non-exudative, or exudative/neovascular), ocular tumors, hereditary retinal degenerations (e.g. retinitis pigmentosa, Stargardt's disease, Usher Syndrome, etc), ocular inflammatory disease (e.g. uveitis), ocular infection (e.g. bacterial, fungal, viral), autoimmune retinitis (e.g. triggered by infection), trauma, diabetic retinopathy, choroidal neovascularization, retinal ischemia, retinal vascular occlusive disease (e.g. branch retinal vein occlusion, central retinal vein occlusion, branch retinal artery occlusion, central retinal artery occlusion, etc.), pathologic myopia, angioid streaks, macular edema (e.g. of any etiology), and/or central serous chorioretinopathy.

As used herein, the term “effective amount” refers to the amount of a composition (e.g., calpain inhibitor) sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment (e.g., compositions of the present invention) to a subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, rectal, by injection (e.g., intravenously, subcutaneously, intratumorally, intraperitoneally, etc.) and the like.

As used herein, the terms “co-administration” and “co-administer” refer to the administration of at least two agent(s) (e.g., multiple calpain inhibitors or a calpain inhibitor with an anti-VEGF therapy or other useful combination) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “pharmaceutical composition” refers to the combination of an active agent (e.g., calpain inhibitor) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. (1975), incorporated herein by reference).

As used herein, the term “pharmaceutically acceptable salt” refers to any salt (e.g., obtained by reaction with an acid or a base) of a compound of the present invention that is physiologically tolerated in the target subject (e.g., a mammalian (e.g., human) subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

### EXAMPLES

### Example 1

### Calpain Inhibition to Treat Eye Disease

This Example describes experiments conducted to test whether calpain activation after retina-RPE separation results in decreased autophagy and increased photoreceptor apoptosis. These experiments were conducted in a well-established in vivo model of retinal detachment and in an in vitro cell culture model. As described below, it was found that Atg5 undergoes up-regulation within 1 day after detachment, similar to other autophagy markers. Calpain 1 reaches peak protein and activity levels at 7 days post-detachment, where autophagy decreases. Calpain inhibitors prolong the period of autophagy, which leads to decreased apoptosis.

### Methods:

### Experimental Model of Retinal Detachment

All experiments were performed in accordance with the ARVO Statement for the Use of Animals in Ophthalmic and Vision Research and the guidelines established by the University Committee on Use and Care of Animals of the University of Michigan. Detachments were created in adult male Brown-Norway rats (300-400 g; Charles River Laboratories, Wilmington, Mass.) as described previously. Briefly, rodents were anesthetized with a 50:50 mix of ketamine/xylazine, and pupils were dilated with topical phenylephrine (2.5%) and tropicamide (1%). A 20-gauge microvitreoretinal blade was used to create a sclerotomy 2 mm posterior to the limbus, carefully avoiding lens damage. A subretinal injector (Glaser, 32-gauge tip; BD Ophthalmic Systems, Franklin Lakes, N.J.) was introduced through the sclerotomy into the vitreous cavity and then through a peripheral retinotomy into the subretinal space. Sodium hyaluronate (10 mg/mL, Healon OVD; Abbott Medical Optics, Uppsala, Sweden) was slowly injected to detach the neurosensory retina from the underlying RPE. In all experiments, approximately one third to one half of the neurosensory retina was detached. Detachments were created in the left eye. The right eye served as the control, with all the steps of the procedure performed except for introduction of the subretinal injector and injection of the sodium hyaluronate. In some experimental eyes, subretinal injection of either Bafilomycin-a (Baf-a, 50 μg; Enzo Life Sciences, Plymouth Meeting, Pa.), 0.2 μg calpeptin (Santa Cruz Biotechnology, Santa Cruz, Calif.) or vehicle (DMSO) was performed at the time of retina-RPE separation. In these experiments, the subretinal sodium hyaluronate was injected as described earlier, immediately followed by additional subretinal injection of the specific agent delivered in a 10 μL volume.

### Cell Culture

The 661W photoreceptor cell line was generously provided by Dr. Muayyad Al-Ubaidi (Department of Cell Biology, University of Oklahoma Health Sciences Center, Oklahoma City, Okla.). The 661W cell line was maintained in Dulbecco's modified Eagle's medium containing 10% fetal bovine serum, 300 mg/L glutamine, 32 mg/L putrescine, 40 μL/L β-mercaptoethanol, and 40 μg/L of both hydrocortisone 21-hemisuccinate and progesterone. The media also contained penicillin (90 units/mL) and streptomycin (0.09 mg/mL). Cells were grown at 37° C. in a humidified atmosphere of 5% CO2 and 95% air.

### Western Blot Analysis

Retinas from experimental eyes with detachment (detached portion of retina only) and control eyes without detachment were dissected from the RPE-choroid, homogenized, and lysed in buffer containing 10 mM HEPES (pH 7.6), 0.5% IgEPal, 42 mM KCl, 1 mM phenylmethylsulfonyl fluoride, 1 mM EDTA, 1 mM EGTA, 1 mM dithiothreitol, 5 mM MgCl2, and one tablet of protease inhibitors per 10 mL buffer (Complete Mini; Roche Diagnostics GmbH, Madison, Wis.). 661W cells were plated at 150,000 cells per well in six-well culture dishes in growth media. After 24 hours, the cells were washed one time with warmed PBS solution and then placed in growth media without fetal bovine serum. Depending on experimental conditions, 500 ng/mL Fas activating antibody (BD Biosciences, San Jose, Calif.) and various calpain inhibitors including MDG-28170 (Sigma, St. Louis, Mo.), MG-101 (Sigma, St. Louis, Mo.), calpeptin (Santa Cruz Biotechnology, Santa Cruz, Calif.) or equal volume of DMSO was added. Calpain inhibitors were added 1 hour prior to the addition of Fas activating antibody. The cells were incubated for various lengths of time and then lysed and homogenized in lysis buffer. The homogenates were incubated on ice and centrifuged at 22,000 g at 4° C. for 60 minutes. The protein concentration of the supernatant was then determined (DC Protein Assay kit; Bio-Rad Laboratories, Hercules, Calif.). The protein samples were separated on SDS-polyacrylamide gels (Tris-HCl Ready Gels; Bio-Rad Laboratories). After electrophoretic separation, the proteins were transferred onto polyvinylidene fluoride membranes (Immobilon-P; Millipore, Billerica, Mass.). Membranes were then immunoblotted with antibodies according to the manufacturer's instructions. The following antibodies were used: LC3b (Cell Signaling, Beverly, Mass.), Atg5 (Abgent, San Diego, Calif.), Atg12 (Cell Signaling, Beverly, Mass.), calpain 1 (Cell Signaling, Beverly, Mass.), alpha-spectrin (Santa Cruz Biotechnology, Santa Cruz, Calif.), actin (Sigma, St. Louis, Mo.), and GAPDH (Abcam, Cambridge, Mass.).

### Caspase Assay and Cell Viability

Caspase 8 activity was measured by a commercially available luminescent cleavage assay kit (Promega, Madison, Wis.). The 661W cells were seeded in 96-well plates at 2,000 cells/well for 24 hours before treatment. Cells were treated with 500 ng/mL Fas-agonistic Jo2 monoclonal antibody and various concentrations of calpeptin or equal volume of DMSO. Caspase 8 activity was measured at 48 hours by incubating the cells with the proluminescent substrate in 96-well plates according to the manufacturer's instructions. Controls included untreated cells and wells with no cells. Luminescence was measured in a plate reader luminometer (Turner Biosystems, Sunnyvale, Calif.).

### Data Analysis

Statistical analysis comparing groups was performed using two-tailed Student's t-tests assuming equal variance. Differences were considered significant at P<0.05.

### Results:

### Atg5 Activity Following Detachment

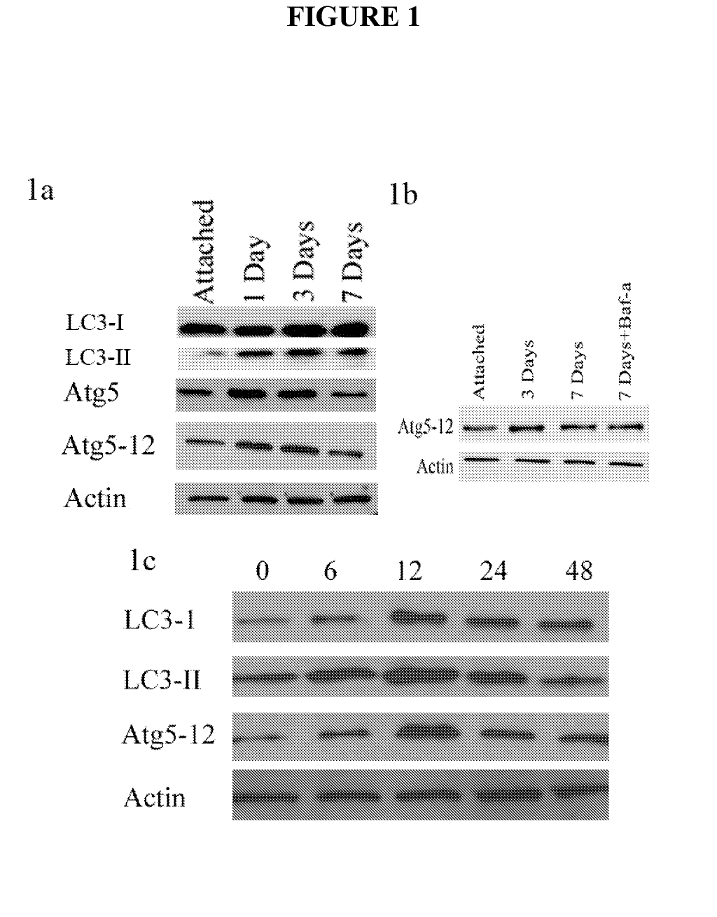

It was first shown that Atg5 protein levels increase following retinal detachment, similar to other biochemical markers of autophagy. Atg5 levels were compared to LC3, an essential component of the autophagosome complex, which has already been well characterized (12). LC3 migrates as two bands on polyacrylamide gel electrophoresis, LC3-I and LC3-II. While the larger LC3-I is cytosolic, LC3-II is a marker of autophagy and is present within the autophagosome. Similar to prior results, LC3-II protein levels were elevated following detachment, peaking at approximately 1-3 days (FIG. 1a). When looking at full length Atg5 and the Atg5-12 complex, the majority state of the protein covalently bound to Atg12, their levels were also elevated at 1 and 3 days, with a slight decrease at 7 days, similar to LC3-II levels (FIG. 1a).

It was previously shown that autophagy is still present at 7 days, but that LC3-II levels decrease in part because LC3-II itself is degraded in the autophagosomes—a concept known as “autophagy flux.” When the degradation of the autophagosome is inhibited by Bafilomycin-a (Baf-a), LC3-II does still remain elevated compared to untreated detached retinas at 7 days (12). Here, when Bafilomycin-a was injected at the time of detachment, there did not appear to be any increase in Atg5-12 levels as compared to detachments alone (FIG. 1b). Therefore, it does appear that Atg5 levels do decrease at 7 days, unrelated to autophagosome turnover.

After determining that Atg5 levels increased following retinal detachment in vivo, it was desired to confirm the results in an in vitro system where 661W cells, a mouse photoreceptor cell line, are activated by a Fas-receptor activating antibody. It has been previously shown that following activation, the 661W cells undergo peak autophagy at about 12 hours and apoptosis at about 48 hours (12, 23). Similar to prior published results, LC3-II peaked at about 12 hours, decreasing to basal levels by about 48 hours (FIG. 1c) (12). Like LC3-II, the Atg5-12 complex also peaked at about 12 hours, confirming peak autophagy activation at about 12 hours after Fas activation (FIG. 1c).

### Calpain 1 Activity Following Detachment

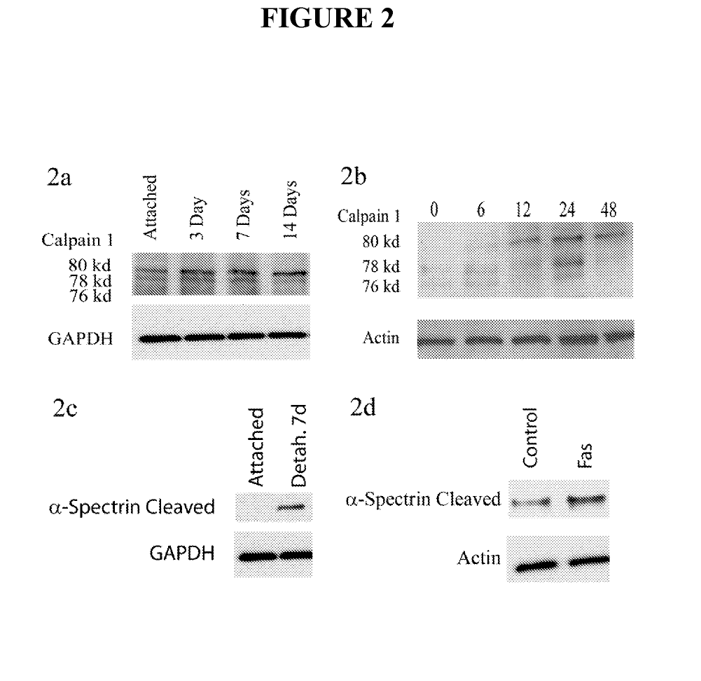

It was sought to confirm that calpains are activated following retinal detachment at time points following peak autophagy activity. This was done first by looking at calpain 1, the calpain responsible for the cleavage and inactivation of Atg5, levels following retinal detachment (13-15). In vivo, there was an increase in full calpain 1 and its autolysis activated subunits after detachment (FIG. 2a). Levels peaked at 7 days and decreased by 14 days. This correlates with FIGS. 1a and 1b, where it was shown that Atg5 levels decrease at 7 days. Calpain 1 levels were also examined in vitro (FIG. 2b). Here, calpain 1 was highest at 24 hours. This again correlates with FIG. 1c, where it was shown a decrease in Atg5 levels between 12 and 24 hours after Fas activation.

After determining calpain 1 levels, it was looked to correlate this with calpain activity. This was done by looking at alpha-spectrin, which when cleaved by calpain 1, forms a breakdown product of 145 kd (15). Compared to eyes with attached retinas, there was a significant increase in calpain 1 specific cleaved alpha-spectrin at 7 days following detachment (FIG. 2c). In addition, there was a significant rise in cleaved alpha-spectrin following Fas activation of 661W cells at 24 hours (FIG. 2d). Thus, the rise in calpain 1 levels at 7 days in vivo and 24 hours in vitro correlated with increased enzyme activity.

### Calpain Inhibition and Prolongation of Autophagy

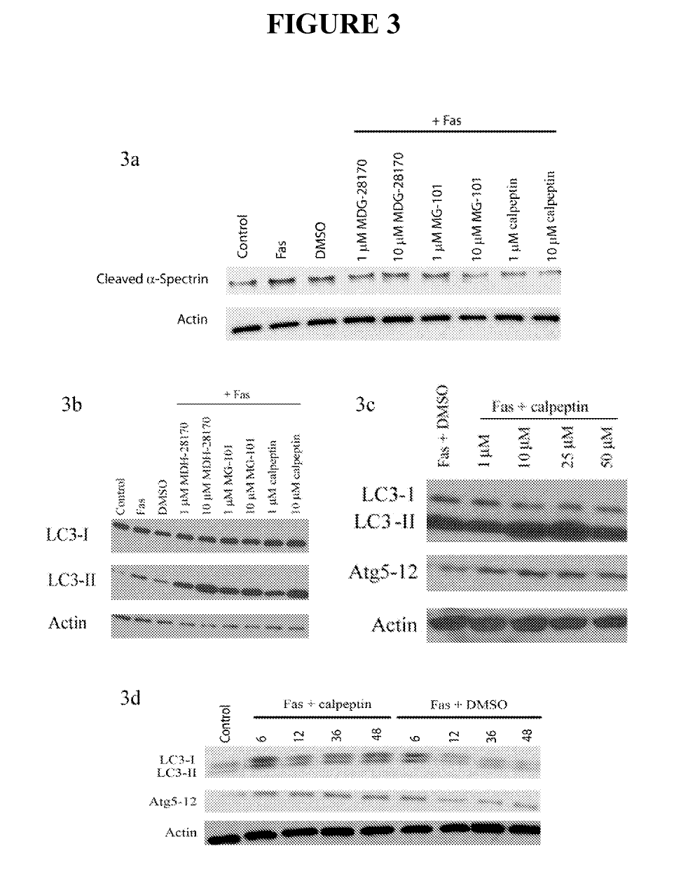

Given that Atg5 levels increased with retinal detachment and that calpain 1 reached peak levels as autophagy was decreasing, next it was examined if calpain inhibitors could prolong autophagy, as calpain inhibitors prevent Atg5 cleavage. A number of pan-calpain inhibitors were tried in an in vitro model, including MDG-28170, MG-101 and calpeptin. All three led to decreased cleaved alpha-spectrin levels at 24 hours in the 661W cells as compared to Fas activation alone. The largest effect, however, was found with calpeptin (FIG. 3a). It was then examined to see if decreased calpain 1 activity was correlated with increased autophagy. There was a significant rise in LC3-II with all the calpain inhibitors at the 24 hour time point, compared to Fas activation alone (FIG. 3b). After determining that calpeptin appeared to inhibit calpain 1 the best, a dose response curve was prepared to determine the optimal concentration for increasing autophagy. Both LC3-II and Atg5-12 peaked at the 25 uM dose of calpeptin (FIG. 3c). It was then examined to see if calpeptin would prolong the two markers of autophagy over time. When compared to cells with Fas activation alone, those cells treated with calpeptin had elongated presence of LC3-II and Atg5-12, indicating prolonged autophagy (FIG. 3d).

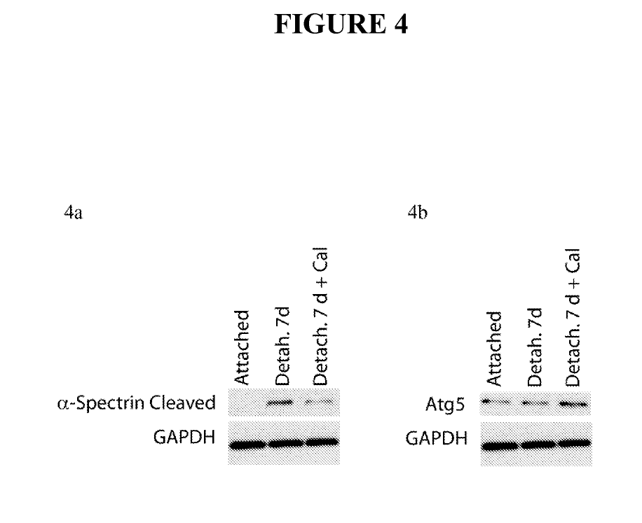

When looking in vivo, similar results were observed. There was less cleaved alpha-spectrin with calpeptin at the 7 day time point, confirming less calpain activity (FIG. 4a). When looking at the autophagy markers, there was significantly more Atg5 at the 7 day time period with calpain inhibition (FIG. 4b). This confirmed the results from the cell culture model that calpeptin is capable of inhibiting calpain in the retina and prolonging autophagy.

### Calpain Inhibition Leads to Decreased Apoptosis

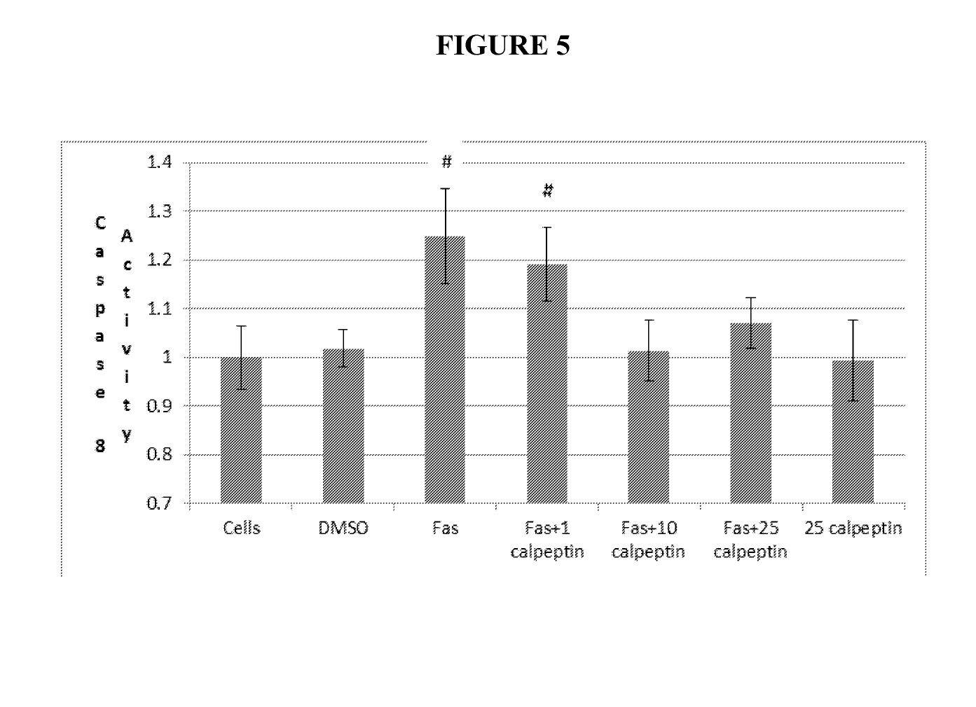

Finally, it was tested to see if inhibiting calpain activity, thereby inhibiting Atg5 cleavage and extending autophagy, led to decreased apoptosis. This was tested ton the 661W cells using a caspase 8 activity assay. Fas activation caused increased caspase 8 activity compared to control cells at 48 hours, similar to prior data (8). Both 10 and 25 uM of calpeptin significantly reduced its activity back to baseline, therefore rescuing the cells from apoptosis (FIG. 5).

Photoreceptor-RPE separation is a common feature of many retinal diseases, and the resultant photoreceptor cell death contributes greatly to the visual morbidity in these patients. Previously, it was shown how both cell survival and cell destructive pathways are activated soon after photoreceptor-RPE separation, leading to a molecular “tug-of-war” pulling the cell towards survival or death, respectively (11-12). In other words, the photoreceptors are triggered to undergo apoptosis as their supply of nutrition is taken away. However, the cells evolved ways of stopping that terminal program for at least a short period of time, in order to recover if the stress is only temporary. This Example shows that inhibition of calpain activation can shift the molecular cascades towards the pro-survival autophagy pathways, with fewer photoreceptors entering the apoptotic cascade.

Strategies being investigated to prolong survival pathways have enormous clinical implications. It is known that after 7 days of detachment, photoreceptors die and visual acuity starts to decrease (9-10). When patients present early with simple retinal detachments, they can be fixed surgically with good visual outcomes. However, in diseases such as AMD, where the detachments are chronic and approximation is not easily achieved, there needs to be ways of prolonging the survival of the remaining photoreceptors if there is any hope of improving visual outcomes.

In this Example, calpain inhibition was examined as a way of prolonging autophagy, a known cell survival pathway up-regulated in many cell types following stress. This was done by following the activity of Atg5, a protein critical to autophagy activation and whose cleavage by calpain 1 resulted in cells aborting autophagy and entering apoptosis in other cell lines (13, 14). It was first shown that Atg5 does rise early after detachment, similar to other markers of autophagy. It was also shown that its levels fall considerably at 7 days, which was confirmed by inhibiting autophagosome turnover with Baf-a. In addition, it was shown that calpain 1 levels peaked at 7 days, which causes the cleavage and inactivation of Atg5 (13, 14). This 7 day time period of decreased Atg5 correlates well with the clinical knowledge that retinal detachments affecting the central macula need to be fixed by 7 days to preserve quality central vision. It is believed that, prior to this study, there has been no explanation as to why vision starts decreasing at this point. This study suggests a possible explanation as to why photoreceptors start decreasing at one week.

This Example has shown that the inhibition of the calpain family, including calpain 1, prolongs autophagy. Both in the in vitro and in vivo system, extended presence of LC3-II and Atg5 were observed with calpain inhibitors. At least in the in vitro system, this extension of autophagy correlated with reduction of apoptosis, rescuing the cells from death.

### REFERENCES

Piccolino F C, de la Longrais R R, Ravera G, et al. The foveal photoreceptor layer and visual acuity loss in central serous chorioretinopathy. Am J Ophthalmol. 2005; 139(1):87-99. Burton T C. Recovery of visual acuity after retinal detachment involving the macula. Trans Am Ophthalmol Soc. 1982; 80:475-497.
Zacks D N, Ha\ddot{}nninen V, Pantcheva M, Ezra E, Grosskreutz C, Miller J W. Caspase activation in an experimental model of retinal detachment. Invest Ophthalmol Vis Sci. 2003; 44(3):1262-1267.
Zacks D N, Zheng Q D, Han Y, Bakhru R, Miller J W. FAS-mediated apoptosis and its relation to intrinsic pathway activation in an experimental model of retinal detachment. Invest Ophthalmol Vis Sci. 2004; 45(12):4563-4569.
Zacks D N, Boehlke C, Richards A L, Zheng Q D. Role of the Fas signaling pathway in photoreceptor neuroprotection. Arch Ophthalmol. 2007; 125(10):1389-1395.
Dunaief J L, Dentchev T, Ying G S, Milam A H. The role of apoptosis in age-related macular degeneration. Arch Ophthal. 2002 November; 120(11):1435-42.
Zacks D N, Boehlke C, Richards A, Zheng A. Role of the Fas-signaling pathway in photoreceptor neuroprotection. Arch Ophthal. 2007 October; 125(10): 1389-95.
Besirli C G, Chinskey N D, Zheng Q D, Zacks D N. (2010) Inhibition of retinal detachment-induced apoptosis in photoreceptors by a small peptide inhibitor of the Fas receptor. IOVS 51, 2177-2184.
Ross W H, Stockl F A. Visual recovery after retinal detachment. Curr Opin Ophthalmol. 2000; 11:191-194.
Ross W H. Visual recovery after macula-off retinal detachment. Eye. 2002; 16:440-446.
Zacks D N, Han Y, Zeng Y, Swaroop A. Activation of signaling pathways and stress-response gene in an experimental model of retinal detachment. IOVS. 2006; 47(4):1691-5.
Besirli C G, Chinskey N D, Zheng Q D, Zacks D N. Autophagy activation in the injured photoreceptor inhibits Fas-mediated apoptosis. IOVS. 2011; 52(7):4193-99.
Yousefi S, Perozzo R, Schmid I, et al. Calpain-mediated cleavage of Atg5 switches autophagy to apoptosis. Nature Cell Bio. 2006; 8(10):1124-32.
Xia H, Zhang L, Chen G, et al. Control of basal autophagy by calpain1 mediated cleavage of Atg5. Autophagy. 2010; 6(1)61-66.
Nakajima E, Hammond K B, Rosales J L, Shearer T R, Azuma M. Calpain, not caspase, is the causative protease for hypoxic damage in cultured monkey retinal cells. IOVS. 2011; 52(10):7059-67.
Sharma A K, Rohrer B. Sustained elevation of intracellular cGMP causes oxidative stress triggering calpain-mediated apoptosis in photoreceptor degeneration. Curr Eye Res. 2007; 32:259-269.
Smith A W, Das A, Guyton M K, Ray S K, Rohrer B, Banik N L. Calpain inhibition attenuates apoptosis of retinal ganglion cells in acute optic neuritis. IOVS. 2011; 52(7):4935-41.
Das A, Garner D P, Del Re A M, et al. Calpeptin provides functional neuroprotection to rat retinal ganglion cells following Ca2_influx. Brain Res. 2006; 1084:146-157.
McKernan D P, Guerin M B, O'Brien C J, Cotter T G. A key role for calpains in retinal ganglion cell death. Invest Ophthalmol Vis Sci. 2007; 48:5420-5430.
Tamada Y, Fukiage C, Mizutani K, et al. Calpain inhibitor, SJA6017, reduces the rate of formation of selenite cataract in rats. Curr Eye Res. 2001; 22:280-285.
Kupina N C, Nath R, Bernath E E, et al. The novel calpain inhibitor SJA6017 improves functional outcome after delayed administration in a mouse model of diffuse brain injury. J Neurotrauma. 2001; 18:1229-1240.
Das A, Sribnick E A, Wingrave J M, et al. Calpain activation in apoptosis of ventral spinal cord 4.1 (VSC4.1) motor neurons exposed to glutamate: calpain inhibition provides functional neuroprotection. J Neurosci Res. 2005; 81:551-562.
Besirli C G, Chinskey N D, Zheng Q D, Zacks D N Inhibition of retinal detachment-induced apoptosis in photoreceptors by a small peptide inhibitor of the fas receptor. IOVS. 2010; 51(4)2177-84.
Koumuro A, Nonaka Y, Hyakkoku K, et al. A novel calpain inhibitor, SNJ-1945, protects neuronal cells from cerebral ischemia-induced damage in mice. Neuroscience. 2008; 157:309-318.
Russo R, Berliocchi L, Adornetto A, et al. Calpain-mediated cleavage of Beclin-1 and autophagy deregulation following retinal ischemic injury in vivo. Cell Death and Disease. 2011; 2, e144.
Kuro M, Katsuhiko Y, Norihisa U, et al. Calpain inhibition restores basal autophagy and suppresses MNU-induced photoreceptor cell death in mice. In Vivo. 2011; 25:617-624.

Although the invention has been described in connection with specific preferred embodiments, it should be understood that the invention as claimed should not be unduly limited to such specific embodiments. Various modification and variation of the described methods and compositions of the invention will be apparent to those skilled in the art without departing from the scope and spirit of the invention. Indeed, various modifications of the described modes for carrying out the invention understood by those skilled in the relevant fields are intended to be within the scope of the following claims. All publications and patents mentioned in the present application are herein incorporated by reference.

We claim:

1. A method for preventing photoreceptor loss in response to retinal detachment comprising, administering a calpain inhibitor to a subject with retinal detachment or suspected of having retinal detachment.

2. Use of a calpain inhibitor for the prevention of photoreceptor apoptosis due to retinal detachment.

3. The method of claim 1 or 2, wherein said subject has age-related macular degeneration.

4. The method of claim 1 or 2, wherein said subject is further administered a VEGF inhibitor.

5. The method of claim 4, wherein said VEGF inhibitor comprises an anti-VEGF antibody.

6. The method of claim 1 or 2, wherein said calpain inhibitor comprises calpeptin.

7. The method of claim 1 or 2, wherein said calpain inhibitor comprises MDG-28170.

8. The method of claim 1 or 2, wherein said calpain inhibitor comprises Calpain Inhibitor I (MG-101).

9. The method of claim 1 or 2, wherein the subject has a disease in which the detachment of the retina is chronic.

10. The method of claim 1 or 2, wherein the calpain inhibitor is administered during or immediately after eye surgery.
